(19) United States
(12) Patent Application Publication
(10) Pub. No.: US 2011/0097414 A1
(43) Pub. Date: Apr. 28, 2011

(54) PHARMACEUTICAL COMPOSITIONS COMPRISING ADSORBATE OF FENOFIBRATE

(76) Inventors: Roshan lal Sandal, Punjab (IN); Vikrant Thakkar, Haryana (IN); Ramakant Kashinath Gundu, Maharashtra (IN); Rahul Sudhakar Dabre, Maharashtra (IN); Narayanan Murali, Tamilnadu (IN); Girish Kumar Jain, Delhi (IN)

(21) Appl. No.: 12/528,627

(22) PCT Filed: Feb. 26, 2008
(86) PCT No.: PCT/IB2008/000414

§ 371 Date: Dec. 27, 2010

(30) Foreign Application Priority Data
Feb. 26, 2007 (IN) 370/MUM/2007
Apr. 20, 2007 (IN) 777/MUM/2007
Apr. 20, 2007 (IN) 778/MUM/2007
Aug. 2, 2007 (IN) 1489/MUM/2007
Aug. 2, 2007 (IN) 1490/MUM/2007
Aug. 2, 2007 (IN) 1491/MUM/2007

(51) Int. Cl. A61K 9/14; A61K 31/235

(52) U.S. Cl. 424/490; 514/543; 424/489

(57) ABSTRACT

The present invention provides a pharmaceutical composition comprising adsorbate of fenofibrate or salt thereof or fenofibrate adsorbed on a pharmaceutically acceptable adsorbent and optionally one or more pharmaceutically acceptable excipients. The invention also relates to processes for the preparation of such compositions.

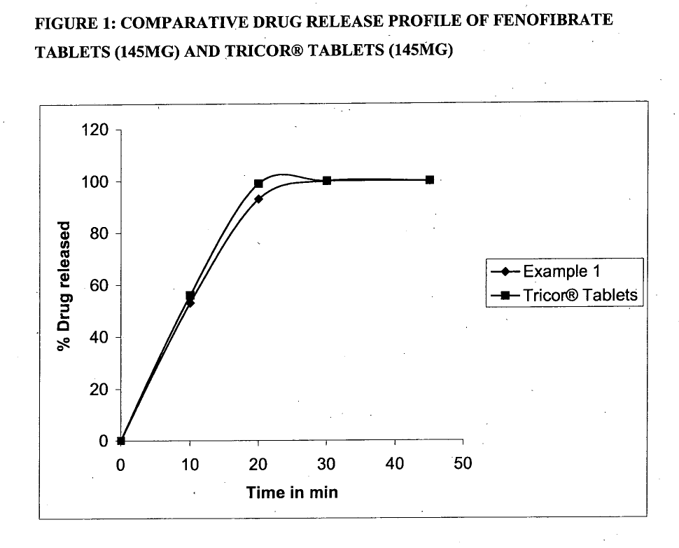

### FIELD OF THE INVENTION

The present invention relates to pharmaceutical compositions comprising adsorbate of fenofibrate or a salt thereof and optionally one or more pharmaceutically acceptable excipients. The invention also relates to pharmaceutical compositions comprising fenofibrate adsorbed on a pharmaceutically acceptable adsorbent and optionally one or more pharmaceutically acceptable excipients. The invention also relates to processes for the preparation of such compositions.

### BACKGROUND OF THE INVENTION

Fenofibrate is a lipid-regulating agent, belongs to the family of fibrates or fibric acid derivatives. It is indicated as an adjunctive therapy to diet for the treatment of adult patients with very high elevations of serum triglyceride levels who are at risk of pancreatitis and who do not respond adequately to dietary control. It is particularly useful for the treatment of adult endogenous hyperlipidemia, hypercholesterolemia and hypertriglyceridemia. It is commercially available as oral capsules containing micronized fenofibrate in the strengths of 67 mg, 134 mg and 200 mg

Fenofibrate is practically insoluble in water and exhibits a low rate of dissolution in aqueous media that results in inadequate bioavailability after oral ingestion. This low rate of dissolution of fenofibrate in aqueous media is also found in gastrointestinal fluids. Chemically, fenofibrate is 2-[4-(4-Chlorobenzoyl)phenoxy]-2-methylpropanoic acid 1-methylethyl ester of formula I. Several methods of increasing the rate of dissolution of drugs having low solubility in water and other aqueous media have been disclosed in the prior art.

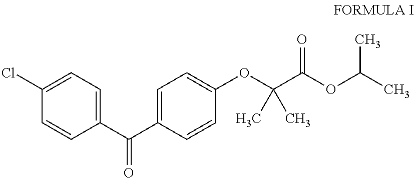

U.S. Pat. Nos. 5,145,684; 6,375,986; 6,969,529; and 6,592,903 disclose nanoparticulate compositions of fenofibrate.

U.S. Pat. Nos. 6,277,405; 6,652,881; 7,037,529; 7,041,319; 6,589,552; 6,531,158 and U.S. Application Nos. 20040057998 and 2004137055 describe micronized fenofibrate compositions.

U.S. Pat. Nos. 4,895,726; 5,880,148 and U.S. Application No. 2004071771 describe co-micronizing the fenofibrate with surface-active agents.

U.S. Pat. No. 6,555,135 describes co-micronized mixture of fenofibrate with pharmaceutically acceptable excipient that is not a surfactant.

U.S. Pat. Nos. 6,074,670 and 6,277,405 describe micronized fenofibrate coated onto hydro soluble carriers with optional surface-active agents.

U.S. Pat. No. 6,828,334 discloses inclusion complex of fenofibrate with cyclodextrins.

U.S. Pat. No. 6,027,747 discloses solid dispersion of fenofibrate.

U.S. Application No. 20040087656 describes fenofibrate of particle size less than 2000 nm with an improved bioavailability.

U.S. Application Nos. 20060222706 and 20060222707 describe fenofibrate in intimate association with menthol or a surfactant mixture.

U.S. Application No. 20030138496 discloses micronized fenofibrate with inert hydro soluble carriers.

The solubility of an active pharmaceutical ingredient influences the bioavailability of the drug. Fenofibrate is a poorly soluble drug. Due to its poor hydrosolubility, the fenofibrate poses problem of low dissolution. It is also poorly absorbed in the digestive tract and consequently its bioavailability is incomplete and irregular. Clearly, there is a need for improved compositions in which the fenofibrate exhibits better dissolution properties.

### SUMMARY OF THE INVENTION

In one general aspect of the invention there is provided a pharmaceutical composition comprising an adsorbate of fenofibrate and optionally, one or more pharmaceutically acceptable excipients.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a pharmaceutical composition comprising fenofibrate adsorbed on a pharmaceutically acceptable adsorbent optionally, along with one or more pharmaceutically acceptable excipients. Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a process for the preparation of an adsorbate of fenofibrate. The process comprising:
a) providing a solution of fenofibrate in one or more organic solvents;
b) adding an adsorbent to the solution of step a) or vice versa; and
c) recovering the adsorbate from mixture of step b) thereof.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a process for the preparation of a pharmaceutical composition of fenofibrate. The process comprising:
a) mixing an adsorbate of fenofibrate with other pharmaceutically acceptable excipients;
b) granulating pre-mix of step a); and
c) converting the granules of step b) into a suitable dosage form.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a pharmaceutical composition comprising an adsorbate of fenofibrate and optionally, one or more pharmaceutically acceptable excipients, wherein the composition exhibits a dissolution profile such that more than 75% of fenofibrate is released within first 30 minutes, wherein the release rate is measured in Apparatus 2 (USP, Dissolution, paddle, 50 rpm) using 1000 ml of 0.05M SLS in water at 37° C.±0.5° C.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a pharmaceutical composition comprising fenofibrate or a pharmaceutically acceptable salt thereof and a pharmaceutically acceptable adsorbent, wherein the adsorbent is pregelatinized starch.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

The term “fenofibrate” as used herein refers to 2-[4-(4-Chlorobenzoyl)phenoxy]-2-methylpropanoic acid 1-methylethyl ester or a salt thereof. The term ‘fenofibrate’ as used herein also refers to non-micronized fenofibrate having a particle size greater than or equal to about 150 μm.

The term “adsorbate” as used herein refers to a physical mixture and/or a complex in which fenofibrate is adhered to or adsorbed on a surface of a pharmaceutically acceptable absorbent.

In another general aspect of the invention there is provided a process for the preparation of a pharmaceutical composition of fenofibrate or a pharmaceutically acceptable salt thereof. The process comprising:
a) dissolving fenofibrate and optionally, one or more binders in one or more organic solvents to form a solution;
b) adsorbing the solution of step a) on pregelatinized starch to obtain an adsorbate of fenofibrate and optionally, drying;
c) layering the adsorbate of fenofibrate of step b) with a solution or dispersion of one or more surfactants; and
d) optionally, adding one or more pharmaceutically acceptable excipients to step c).

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a pharmaceutical composition comprising fenofibrate or a pharmaceutically acceptable salt thereof and a pharmaceutically acceptable adsorbent, wherein the adsorbent is pregelatinized starch and wherein the composition exhibits a dissolution profile such that more than 75% of fenofibrate is released within first 30 minutes when the release rate is measured in Apparatus 2 (USP, Dissolution, paddle, 50 rpm) using 1000 ml of 0.05M SLS in water at 37° C.±0.5° C.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a process for the preparation of a pharmaceutical composition comprising an adsorbate of fenofibrate or a pharmaceutically acceptable salt thereof. The process comprising:
a) dissolving fenofibrate and optionally, one or more binders in one or more organic solvents to form a solution;
b) adsorbing the solution of step a) on one or more pharmaceutically acceptable adsorbents to obtain an adsorbate of fenofibrate and optionally, drying;
c) layering the adsorbate of fenofibrate of step b) with a solution or dispersion of one or more surfactants; and
d) optionally, adding one or more pharmaceutically acceptable excipients to step c).

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a pharmaceutical composition of fenofibrate or a salt thereof comprising non-micronized fenofibrate, pharmaceutically acceptable adsorbent and polyethylene glycol or a derivative thereof.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

The term ‘non-micronized fenofibrate’ as used herein refers to fenofibrate having a particle size greater than or equal to about 50 μm and fenofibrate is not subjected to any comminution techniques that are well known to person skilled in the art and include but not limited to milling, spray drying, or high pressure homogenization.

In another general aspect of the invention there is provided a pharmaceutical composition of fenofibrate or a salt thereof comprising non-micronized fenofibrate, pharmaceutically acceptable adsorbent and polyethylene glycol or a derivative thereof, wherein the adsorbent and polyethylene glycol or a derivative thereof is alternately coated with non-micronized fenofibrate and a surfactant.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a process for the preparation of a pharmaceutical composition of fenofibrate or a salt thereof, the process comprising:
a) preparing a solution of fenofibrate comprising non-micronized fenofibrate and optionally, one or more pharmaceutically acceptable excipients;
b) preparing a solution of a surfactant comprising one or more surfactants and optionally, one or more pharmaceutically acceptable excipients; and
c) coating the solution of step a) and step b) alternately on a pharmaceutically acceptable adsorbent and polyethylene glycol (PEG) or a derivative thereof.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a pharmaceutical composition of fenofibrate or a salt thereof comprising fenofibrate and a pharmaceutically acceptable adsorbent, wherein the adsorbent is alternately coated with a non-micronized fenofibrate and one or more surfactants.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a process for the preparation of a pharmaceutical composition of fenofibrate or a salt thereof, the process comprising:
a) preparing a solution of fenofibrate comprising fenofibrate and optionally, one or more pharmaceutically acceptable excipients;
b) preparing a solution of surfactant comprising one or more surfactants and optionally, one or more pharmaceutically acceptable excipients;
c) coating the solution of step a) and step b) alternately on a pharmaceutically acceptable adsorbent.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

In another general aspect of the invention there is provided a pharmaceutical composition of fenofibrate or a pharmaceutically acceptable salt thereof which when administered to human subjects in the fed state at a dose of 145 mg exhibits (a) the mean area under the 96 hour AUC curve in the range from about 56.02 to about 268.23 (μg*hr/ml); (b) the mean area under the AUC curve extrapolated to infinite time in the range from about 59.07 to about 291.33 (μg*hr/ml); and (c) the maximum plasma concentration in the range from about 3.886 to about 20.703 μg/ml.

Embodiments of the pharmaceutical composition may include one or more of the following features. For example, the pharmaceutically acceptable excipients may include one or more of fillers, binders, lubricants, sweeteners, coloring and flavoring agents, glidants, disintegrants, and the like.

The details of one or more embodiments of the inventions are set forth in the description below. Other features, objects and advantages of the inventions will be apparent from the description and claims.

### DETAILED DESCRIPTION OF THE INVENTION

The present inventors while working on fenofibrate formulation have noticed that when fenofibrate is adsorbed on a suitable adsorbent, it adheres to various interparticle and intraparticle pores present on the surface of adsorbent that provides large exposed surface area for drug loading resulting in increased solubility of fenofibrate in aqueous fluids which, in turn, leads to a significant increase in percent drug release of fenofibrate and hence increased bioavailability. Pregelatinised starch may be used as an adsorbent. Also, it was found by the present inventors that the composition of fenofibrate comprising fenofibrate adsorbed on pregelatinized starch exhibits similar dissolution profile as that of Tricor® tablets (commercially available fenofibrate tablets).

The present inventors also have noticed that when pharmaceutically acceptable adsorbent and polyethylene glycol (PEG) or a derivative thereof is alternately coated with a layer of non-micronized fenofibrate and a surfactant, it resulted in a significant increase in the solubility of fenofibrate in aqueous fluids which, in turn, leads to significant increase in percent drug release of fenofibrate and hence increased bioavailability.

The present inventors have further noticed that when pharmaceutically acceptable adsorbent and PEG or a derivative thereof is alternately coated with a layer of non-micronized fenofibrate and a surfactant, the surfactant remains available with fenofibrate for longer duration of time when compared to fenofibrate formulation wherein pharmaceutically acceptable adsorbent and PEG or a derivative thereof is coated with a single layer of non-micronized fenofibrate followed by a single layer of surfactant.

The term ‘fenofibrate’ as used herein refers to non-micronized fenofibrate having a particle size greater than or equal to about 150 μm.

The term ‘non-micronized fenofibrate’ as used herein refers to fenofibrate having a particle size greater than or equal to about 50 μm and fenofibrate is not subjected to any comminution techniques that are well known to a person skilled in the art and include but not limited to milling, spray drying, high pressure homogenization.

The term ‘adsorbate’ as used herein refers to a physical mixture and/or a complex in which fenofibrate is adhered to or adsorbed on a surface of a pharmaceutically acceptable adsorbent.

The adsorbate of fenofibrate contains fenofibrate in an amount of from about 1% to about 70% by weight and pharmaceutically acceptable adsorbent from about 30% to about 99% by weight.

Suitable pharmaceutically acceptable adsorbents may be one or more of colloidal silicon dioxide, calcium silicate, magnesium aluminum silicate, porous ceramics, polypropylene foams, cellulose, cellulose derivatives, polyols, starches, pre-gelatinized starches, starch derivatives, modified starches, dextrins, maltodextrins, polydextroses, dextroses, calcium carbonate, calcium phosphate, and calcium sulfate. In general, the solution of fenofibrate can be prepared with a solvent in which the fenofibrate is soluble while the adsorbent should be not soluble or only sparingly soluble in this solvent. The term “soluble” and “sparingly soluble” as used herein refers to descriptive terms of solubility as per United States Pharmacopoeia (USP 29/NF 24).

In general, the adsorbate can be recovered from the suspension by any suitable means, such as removal of the solvent. The removal of the solvent can be carried out by means of drying the mixture with or without vacuum, freeze-drying or lyophilization. The drying may include evaporation and/or distillation or any other means known to a skilled artisan for removal of the solvent from mixture.

The pharmaceutical composition of the present invention can be prepared by dissolving fenofibrate in a suitable solvent and adding an adsorbent to fenofibrate solution. The wet mass thus obtained may be dried, blended with other pharmaceutically acceptable excipients and granulated with a binder. The granules may be dried, sized, mixed with other pharmaceutically acceptable excipients, lubricated and compressed.

The fenofibrate solution can be adsorbed on pregelatinized starch using conventional methods known in the art and include, but not limited to, Glatt processor, rapid mixer granulator (RMG), and the like.

The pharmaceutical composition of the present invention can be prepared by dissolving fenofibrate and binder in a suitable solvent and adsorbing the obtained solution on pregelatinized starch using Glatt processor. The wet mass thus obtained may be dried and the obtained adsorbate of fenofibrate may be layered with a surfactant solution using Glatt processor. The Wet mass may be dried, blended with other pharmaceutically acceptable excipients, lubricated and compressed to form a tablet. The obtained tablets can be optionally coated with aqueous dispersion of opadry.

The pharmaceutical composition of the present invention comprising 145 mg of fenofibrate, and pregelatinized starch as an adsorbent can be used to make formulations such as tablets or capsules. The tablets comprising about 145 mg fenofibrate were administered orally to human subjects in a fed state and the pharmacokinetics based on the plasma concentration of fenofibric acid was determined and is shown in Table 11. The composition of the present invention was found to be bioequivalent to commercially available Tricor® tablets.

The geometric mean of the ratio of the AUC 0-96h for the formulation of the present invention administered orally to a group of human subjects in a fasted state versus the AUC 0-96h of Tricor® 145 mg tablets administered orally to the group of human subjects in the fasted state is about 0.80 to about 1.25, preferably about 1.

Similarly, the geometric mean of the ratio of the AUC.0-infinity for a formulation of the present invention when orally administered to a group of human subjects in a fed state versus the AUC. 0-infinity of Tricor® 145 mg tablets administered orally to the group of human subjects in the fed state is about 0.80 to about 1.25, preferably about 1.

The geometric mean of the ratio of Cmax for the formulation of the present invention administered orally to a group of human subjects in a fed state versus the Cmax for Tricor® 145 mg tablets administered orally to the group of human subjects in the fed state is about 0.80 to about 1.25, preferably about 1.

The pharmaceutical composition of the present invention is meant for oral administration and can be present in the form of a tablet, a capsule, powder, disc, a caplet, granules and pellets.

Examples of binders include, but not limited to, polyvinylpyrrolidone, ethylcellulose, and low molecular weight hydroxypropyl methylcellulose.

Suitable organic solvents include methanol, ethanol, isopropanol, acetone, ether, chloroform, dimethyl sulfoxide (DMSO), dimethylformamide (DMF), and methylene chloride.

The surfactants which may be used in the process of the present invention include, but not limited to, amphoteric, non-ionic, cationic or anionic surfactants. Examples of such surfactants include sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, Poloxamer®, etc. The mixtures of surfactants are also suitable.

The one or more pharmaceutically acceptable excipients include one or more of fillers, binders, lubricants, disintegrants, and glidants.

Suitable fillers may be one or more of microcrystalline cellulose, silicified microcrystalline cellulose, mannitol, calcium phosphate, calcium sulfate, kaolin, dry starch, powdered sugar, and the like.

Suitable binders may be one or more of povidone, starch, stearic acid, gums, hydroxypropylmethyl cellulose, and the like.

Suitable lubricants may be one or more of, magnesium stearate, zinc stearate, calcium stearate, stearic acid, sodium stearyl fumarate, hydrogenated vegetable oil, glyceryl behenate, and the like.

Suitable disintegrants may be one or more of starch, croscarmellose sodium, crospovidone, sodium starch glycolate, and the like.

Suitable glidants may be one or more of colloidal silicon dioxide, talc or cornstarch, and the like.

The present invention is further illustrated by the following examples which are provided merely to be exemplary of the invention and do not limit the scope of the invention. Certain modifications and equivalents will be apparent to those skilled in the art and are intended to be included within the scope of the present invention.

### EXAMPLES

The composition and dissolution data of batches is provided in table 1-13. The following formulations are representatives of the preferred compositions of the present invention. The preparation method of dosage form is detailed below.

### Example-I

TABLE 1
Composition of Fenofibrate Tablets (48 mg, 145 mg)

| Sr. No. | Ingredients | Qty/tablet (% w/w) |
|---|---|---|
| | Part-I | |
| 1 | Fenofibrate | 15-50 |
| 2 | Calcium silicate | 5-70 |
| 3 | Acetone | q.s. |
| | Part-II | |
| 4 | Lactose | 20-70 |
| 5 | Silicified microcrystalline cellulose | 5-70 |
| 6 | Crospovidone | 1-6 |
| 7 | Povidone | 0.1-10 |
| 8 | Purified water | q.s. |
| 9 | Magnesium stearate | 0.1-3 |
| 10 | Opadry | 0.5-5 |

Procedure: Fenofibrate was dissolved in a sufficient quantity of acetone to get a clear solution. Calcium silicate was added to the clear fenofibrate solution under stirring. The wet mass thus obtained was tray dried overnight in an oven at 35-40° C. The dried mass was sieved and blended with presifted lactose, microcrystalline cellulose, and crospovidone in a rapid mixer granulator. The above blend was granulated with povidone solution in a rapid mixer granulator. The granules were dried, milled and blended with presifted crospovidone, lubricated with magnesium stearate and lubricated blend was compressed into tablets. The compressed tablets were coated with aqueous dispersion of Opadry.

### Example-2

TABLE 2
Composition of Fenofibrate Tablets (48 mg, 145 mg)

| Sr. No. | Ingredients | Qty/tablet (% w/w) |
|---|---|---|
| | Part-I | |
| 1 | Fenofibrate | 15-50 |
| 2 | Calcium silicate | 5-70 |
| 3 | Acetone | q.s. |
| | Part-II | |
| 4 | Lactose | 20-70 |
| 5 | Silicified microcrystalline cellulose | 5-70 |
| 6 | Crospovidone | 1-6 |
| 7 | Povidone | 0.1-10 |
| 8 | Sodium lauryl sulfate | 0.1-10 |
| 9 | Docusate sodium | 0.05-5 |
| 10 | Purified water | q.s. |
| 11 | Magnesium stearate | 0.1-3 |
| 12 | Opadry | 0.5-5 |

Procedure: Fenofibrate was dissolved in a sufficient quantity of acetone to get a clear solution. Calcium silicate was added to the clear fenofibrate solution under stirring. The wet mass thus obtained was tray dried overnight in an oven at 35-40° C. The dried mass was sieved and blended with presifted lactose, microcrystalline cellulose, and crospovidone in a rapid mixer granulator. The above, blend was granulated with povidone solution containing sodium lauryl sulfate and docusate sodium in a rapid mixer granulator. The granules were dried, milled and blended with presifted crospovidone, lubricated with magnesium stearate and lubricated blend was compressed into tablets. The compressed tablets were coated with aqueous dispersion of Opadry.

TABLE 3
Dissolution data of Fenofibrate tablets (145 mg)

| Time | % drug released | % drug released |
|---|---|---|
| (min) | (Example-I) | (Example-II) |
| 10 | 65 | 65 |
| 20 | 75 | 72 |
| 30 | 83 | 87 |
| 45 | 98 | 97 |

Table 3 provides the dissolution data for fenofibrate tablets (145 mg) prepared as per the formula given in Table 1 and 2. For determination of drug release rate, USP Type 2 Apparatus (rpm 50) was used, wherein 1000 ml of 0.05M SLS in water at 37° C. ± 0.5° C. was used as a medium.

### Example-3

TABLE 4
Composition of Fenofibrate Tablets (48 mg, 145 mg)

| Sr. No. | Ingredients | Qty/tablet (% w/w) |
|---|---|---|
| | Part I | |
| 1. | Fenofibrate | 20-70 |
| 2. | PVP K-30 | 1-30 |
| 3. | Pregelatinized starch | 5-70 |
| 4. | Acetone | q.s |
| 5. | IPA | q.s |
| | Part II | |
| 6. | Docusate Sodium | 0.05-5 |
| 7. | Sodium Lauryl Sulphate | 0.1-10 |
| 8. | Acetone | q.s |
| 9. | Water | q.s |
| | Part III Extragranular | |
| 10 | Microcrystalline Cellulose | 10-50 |
| 11 | Crospovidone | 1-10 |
| 12 | Magnesium stearate | 0.1-2 |
| 13 | Opadry | 0.5-5 |

Procedure: Fenofibrate and polyvinylpyrrolidone (PVP K-30) were dissolved in a sufficient quantity of acetone and isopropyl alcohol (IPA) to get a clear solution. The fenofibrate solution was adsorbed on pregelatinized starch using a Glatt processor. The wet mass thus obtained was tray dried overnight in an oven at 35-40° C. (step 1). Docusate sodium and sodium lauryl sulphate were dissolved in a sufficient quantity of acetone and water mixture (step 2). The obtained solution was layered onto the dried mass obtained in step 1 using a Glatt processor and then dried. The dried mass was sieved through ASTM 30 mesh and blended with presifted microcrystalline cellulose and crospovidone in a rapid mixer granulator. The above blend was lubricated with magnesium stearate and compressed to form tablets using a suitable tooling. The compressed tablets were coated with aqueous dispersion, of Opadry.

TABLE 5
Dissolution data of Fenofibrate tablets (145 mg)
and Tricor ® Tablets (145 mg)

| Time | % drug released | % drug released |
|---|---|---|
| (min) | (Example-3) | (Tricor ® Tablets) |
| 10 | 53 | 56 |
| 20 | 93 | 99 |
| 30 | 100 | 100 |
| 45 | 100 | 100 |

Table 5 provides the dissolution data for fenofibrate tablets (145 mg) prepared as per the formula given in Table 4 and commercially available Tricor ® Tablets. For determination of drug release rate, USP Type 2 Apparatus (rpm 50) was used, wherein 1000 ml of 0.05M SLS in water at 37° C. ± 0.5° C. was used as a medium.

### Example-4

TABLE 6
Composition of Fenofibrate Tablets (48 mg, 145 mg)

| Sr. No. | Ingredients | Qty/tablet (% w/w) |
|---|---|---|
| | Part I | |
| 1. | Fenofibrate | 20-70 |
| 2. | PVP K-30 | 1-30 |
| 3. | Calcium silicate | 5-70 |
| 4. | Acetone | q.s |
| 5. | IPA | q.s |
| | Part II | |
| 6. | Docusate Sodium | 0.05-5 |
| 7. | Sodium Lauryl Sulphate | 0.1-10 |
| 8. | Acetone | q.s |
| 9. | Water | q.s |
| | Part III Extragranular | |
| 10 | Microcrystalline Cellulose | 10-50 |
| 11 | Crospovidone | 1-10 |
| 12 | Magnesium stearate | 0.1-2 |
| 13 | Opadry | 0.5-5 |

Procedure: Fenofibrate and polyvinylpyrrolidone (PVP K-30) were dissolved in a sufficient quantity of acetone and isopropyl alcohol (IPA) to get a clear solution. The fenofibrate solution was adsorbed on calcium silicate using a Glatt processor. The wet mass thus obtained was tray dried overnight in an oven at 35-40° C. (step 1). Docusate sodium and sodium lauryl sulphate were dissolved in a sufficient quantity of acetone and water mixture (step 2). The obtained solution was layered onto the dried mass obtained in step 1 using a Glatt processor and then dried. The dried mass was sieved through ASTM 30 mesh and blended with presifted microcrystalline cellulose and crospovidone in a rapid mixer granulator. The above blend was lubricated with magnesium stearate and compressed to form tablets using a suitable tooling. The compressed tablets were coated with aqueous dispersion of Opadry.

### Example-5

TABLE 7
Composition of Fenofibrate Tablets (48 mg, 145 mg)

| Sr. No. | Ingredients | Qty/tablet (% w/w) |
|---|---|---|
| | Part I | |
| 1. | Fenofibrate | 20-70 |
| 2. | PVP K-30 | 1-30 |
| 3. | Pregelatinized starch | 5-70 |
| 4. | Acetone | q.s |
| 5. | IPA | q.s |
| | Part II | |
| 6. | Docusate Sodium | 0.05-5 |
| 7. | Sodium Lauryl Sulphate | 0.1-10 |
| 8. | Acetone | q.s |
| 9. | Water | q.s |
| | Part III Extragranular | |
| 10 | Microcrystalline Cellulose | 10-50 |
| 11 | Crospovidone | 1-10 |
| 12 | Magnesium stearate | 0.1-2 |
| 13 | Opadry | 0.5-5 |

Procedure: Fenofibrate and polyvinylpyrrolidone (PVP K-30) were dissolved in a sufficient quantity of acetone and isopropyl alcohol (IPA) to get a clear solution. The fenofibrate solution was adsorbed on pregelatinized starch using a Glatt processor. The wet mass thus obtained was tray dried overnight in an oven at 35-40° C. (step 1). Docusate sodium and sodium lauryl sulphate were dissolved in a sufficient quantity of acetone and water mixture (step 2). The obtained solution was layered onto the dried mass obtained in step 1 using a Glatt processor and then dried. The dried mass was sieved through ASTM 30 mesh and blended with presifted microcrystalline cellulose and crospovidone in a rapid mixer granulator. The above blend was lubricated with magnesium stearate and compressed to form tablets using a suitable tooling. The compressed tablets were coated with aqueous dispersion of Opadry.

### Example-6

TABLE 8
Composition of Fenofibrate Tablets (48 mg, 145 mg)

| Sr. No. | Ingredients | Qty/tablet (% w/w) |
|---|---|---|
| | Part I | |
| 1. | Fenofibrate | 20-70 |
| 2. | PVP K-30 | 1-30 |
| 3. | Amorphous silicon dioxide | 5-70 |
| 4. | Acetone | q.s |
| 5. | IPA | q.s |
| | Part II | |
| 6. | Docusate Sodium | 0.05-5 |
| 7. | Sodium Lauryl Sulphate | 0.1-10 |
| 8. | Acetone | q.s |
| 9. | Water | q.s |
| | Part III Extragranular | |
| 10 | Microcrystalline Cellulose | 10-50 |
| 11 | Crospovidone | 1-10 |
| 12 | Magnesium stearate | 0.1-2 |
| 13 | Opadry | 0.5-5 |

Procedure: Fenofibrate and polyvinylpyrrolidone (PVP K-30) were dissolved in a sufficient quantity of acetone and isopropyl alcohol (IPA) to get a clear solution. The fenofibrate solution was adsorbed on amorphous silicon dioxide using a Glatt processor. The wet mass thus obtained was tray dried overnight in an oven at 35-40° C. (step 1). Docusate sodium and sodium lauryl sulphate were dissolved in a sufficient quantity of acetone and water mixture (step 2). The obtained solution was layered onto the dried mass obtained in step 1 using a Glatt processor and dried. The dried mass was sieved through ASTM 30 mesh and blended with presifted microcrystalline cellulose and crospovidone in a rapid mixer granulator. The above blend was lubricated with magnesium stearate and compressed to form tablets using a suitable tooling. The compressed tablets were coated with aqueous dispersion of Opadry.

TABLE 9
Dissolution data of Fenofibrate tablets (145 mg)

| Time | % drug released | % drug released |
|---|---|---|
| (min) | (Example-4) | (Example-5) |
| 10 | 45 | 50 |
| 20 | 70 | 93 |
| 30 | 85 | 100 |
| 45 | 98 | 100 |

Table 9 provides the dissolution data for fenofibrate tablets (145 mg) prepared as per the formula given in Table 6 and 7. For determination of drug release rate, USP Type 2 Apparatus (rpm 50) was used, wherein 1000 ml of 0.05M SLS in water at 37° C. ± 0.5° C. was used as a medium.

TABLE 10
Fed Biostudy data of 145 mg Fenofibrate Tablets (Sample A) against
commercially available Tricor ® Tablets

| | Example 1 | | Tricor ® Tablets | |
|---|---|---|---|---|
| | (145 mg Fenofibrate Tablets) | | (145 mg) | |
| Pharmacokinetic | | Geometric | | Geometric |
| Parameters | Mean | mean | Mean | mean |
| Cmax | 9.1 | 8.3 | 9.46 | 9.13 |
| AUC 0-t | 129.33 | 117.02 | 130.66 | 121.64 |
| AUC 0-∞ | 136.46 | 123.01 | 138.02 | 127.77 |

Cmax = Maximum plasma concentration in μg/ml
AUC (o-t) = Area under the plasma concentration time curve from time 0 to t (t = 96 hrs).
AUC (o-∞ [infinity]) = Area under the plasma concentration time curve from time 0 to infinity.

The pharmaceutical composition of the present invention i.e. Sample A (145 mg) was found to be bioequivalent to the innovator tablet (Tricor® Tablets 145 mg).

### Example 7

TABLE 11
Composition of Fenofibrate Tablets

| Sr. No. | Ingredients | Qty/tablet (% w/w) |
|---|---|---|
| | Part I | |
| 1. | Fenofibrate* | 20-70 |
| 2. | Pregelatinized starch | 5-50 |
| 3. | PEG 6000 | 5-50 |
| 4. | Poloxamer | 5-50 |
| 5. | Povidone K-30 | 1-30 |
| | Part II | |
| 6. | Docusate Sodium | 0.05-5 |
| 7. | Sodium Lauryl Sulphate | 0.1-10 |
| | Part III | |
| 8. | Prosolv SMCC 90 | 10-50 |
| 9. | Crospovidone | 1-10 |
| 10. | Magnesium Stearate | 0.1-2 |

*Fenofibrate was non-micronized having a particle size greater than or equal to 50 μm

Procedure: Fenofibrate and Povidone K-30 were dissolved in a suitable solvent to get a clear solution. Pregelatinized starch and PEG 6000 were alternately coated with 33% w/w of total poloxamer solution and 33% w/w of total fenofibrate solution till 100% w/w of both the solutions were layered. The obtained granules were further coated with a solution of docusate sodium and sodium lauryl sulphate and dried. The obtained granules were mixed with Prosolv SMCC 90 and crospovidone followed by lubricating the granules with magnesium stearate. The lubricated granules were compressed into tablets using a suitable tooling and optionally coated with aqueous dispersion of opadry.

### Example 8

TABLE 12
Composition of Fenofibrate Tablets

| Sr. No. | Ingredients | Qty/tablet (% w/w) |
|---|---|---|
| | Part I | |
| 1. | Fenofibrate* | 20-70 |
| 2. | Pregelatinized starch | 5-70 |
| 3. | PEG 6000 | 5-50 |
| 4. | Poloxamer | 5-50 |
| 5. | Povidone K-30 | 1-30 |
| | Part II | |
| 6. | Docusate Sodium | 0.05-5 |
| 7. | Sodium Lauryl Sulphate | 0.1-10 |
| | Part III | |
| 8. | Prosolv SMCC 90 | 10-50 |
| 9. | Crospovidone | 1-10 |
| 10. | Magnesium Stearate | 0.1-2 |

*Fenofibrate was non-micronized having a particle size greater than or equal to 50 μm

Procedure: Fenofibrate and Povidone K-30 were dissolved in a suitable solvent to get a clear solution. Pregelatinized starch and PEG 6000 were alternately coated with 25% w/w of total poloxamer solution and 25% w/w of total fenofibrate solution till 100% w/w of both the solutions were layered. The obtained granules were further coated with a solution of docusate sodium and sodium lauryl sulphate and dried. The obtained granules were mixed with Prosolv SMCC 90 and crospovidone followed by lubricating the granules with magnesium stearate. The lubricated granules were compressed into tablets using a suitable tooling and optionally coated with aqueous dispersion of opadry.

### Example 9

TABLE 13
Composition of Fenofibrate Tablets

| Sr. No. | Ingredients | Qty/tablet (% w/w) |
|---|---|---|
| | Part I | |
| 11. | Fenofibrate | 20-70 |
| 12. | Pregelatinized starch | 5-70 |
| 13. | Poloxamer | 5-50 |
| 14. | Povidone K-30 | 1-30 |
| | Part II | |
| 15. | Docusate Sodium | 0.05-5 |
| 16. | Sodium Lauryl Sulphate | 0.1-10 |
| | Part III | |
| 17. | Prosolv SMCC 90 | 10-50 |
| 18. | Crospovidone | 1-10 |
| 19. | Magnesium Stearate | 0.1-2 |

Procedure: Fenofibrate and Povidone K-30 were dissolved in a suitable solvent to get a clear solution. Pregelatinized starch was alternately coated with 33% w/w of total poloxamer solution and 33% w/w of total fenofibrate solution till 100% w/w of both the solutions were layered. The obtained granules were further coated with a solution of docusate sodium and sodium lauryl sulphate and dried. The obtained granules were mixed with Prosolv SMCC 90 and crospovidone followed by lubricating the granules with magnesium stearate. The lubricated granules were compressed into tablets using a suitable tooling and optionally coated with aqueous dispersion of opadry.

While the present invention has been described in terms of its specific embodiments, certain modifications and equivalents will be apparent to those skilled in the art and are intended to be included within the scope of the present invention.

1-45. (canceled)

46. A pharmaceutical composition comprising adsorbate of fenofibrate or fenofibrate adsorbed on a pharmaceutically acceptable adsorbent, and optionally, one or more pharmaceutically acceptable excipients.

47. The pharmaceutical composition according to claim 46, comprising fenofibrate in an amount of from about 1% to about 70% by weight and a pharmaceutically acceptable adsorbent from about 30% to about 99% by weight.

48. The pharmaceutical composition according to claim 46, wherein the composition is in the form of a tablet, capsule, powder, disc, caplet, granules, or pellets.

49. The pharmaceutical composition according to claim 46, wherein the pharmaceutically acceptable adsorbent comprises one or more of colloidal silicon dioxide, calcium silicate, magnesium aluminum meta silicate, porous ceramics, polypropylene foams, cellulose, cellulose derivatives, polyols, starches, pre-gelatinized starches, starch derivatives, modified starches, dextrins, maltodextrins, polydextroses, dextroses, calcium carbonate, calcium phosphate, and calcium sulfate.

50. A process for preparation of an adsorbate of fenofibrate according to claim 46, the process comprising: (a) providing a solution of fenofibrate in one or more organic solvents; (b) adding an adsorbent to the solution of step (a) or vice versa; and (c) recovering the adsorbate from mixture of step (b).

51. The process according to claim 50, wherein the organic solvent comprises one or more of methanol, ethanol, isopropanol, acetone, ether, chloroform, dimethyl sulfoxide, dimethylformamide, and methylene chloride.

52. A process for preparation of a pharmaceutical composition of fenofibrate according to claim 46, the process comprising: (a) mixing an adsorbate of fenofibrate with other pharmaceutically acceptable excipients; (b) granulating pre-mix of step (a); and (c) converting the granules of step (b) into a suitable dosage form.

53. The pharmaceutical composition according to claim 46, wherein the formulation exhibits a dissolution profile such that more than 75% of fenofibrate is released within first 30 minutes when the release rate is measured in Apparatus 2 (USP, Dissolution, paddle, 50 rpm) using 1000 ml of 0.05 M SLS in water at 37° C.±0.5° C.

54. The pharmaceutical composition according to claim 49, wherein the pharmaceutically acceptable adsorbent is pre-gelatinized starch.

55. The pharmaceutical composition according to claim 9, wherein the fenofibrate is non-micronized and has a particle size greater than or equal to about 150 μm.

56. A process for the preparation of a pharmaceutical composition according to claim 54, the process comprising: (a) dissolving fenofibrate and optionally, one or more binders in one or more organic solvents to form a solution; (b) adsorbing solution of step (a) on pre-gelatinized starch to obtain an adsorbate of fenofibrate and optionally drying; (c) layering the adsorbate of fenofibrate of step (b) with a solution or dispersion of one or more surfactants; and (d) optionally, adding one or more pharmaceutically acceptable excipients to step (c).

57. The process according to claim 56, wherein the binder comprises one or more of polyvinylpyrrolidone, ethylcellulose, and low molecular weight hydroxypropyl methylcellulose.

58. The process according to claim 56, wherein the organic solvent comprises one or more of methanol, ethanol, isopropanol, acetone, ether, chloroform, dimethylsulfoxide, dimethylformamide, and methylene chloride.

59. The process according to claim 56, wherein the surfactants comprises one or more of amphoteric, non-ionic, cationic or anionic surfactants.

60. The process according to claim 56, wherein the pharmaceutically acceptable excipients comprises one or more of fillers, lubricants, disintegrants, and glidants.

61. The pharmaceutical composition according to claim 54, wherein the formulation exhibits a dissolution profile such that more than 75% of fenofibrate is released within first 30 minutes when the release rate is measured in Apparatus 2 (USP, Dissolution, paddle, 50 rpm) using 1000 ml of 0.05 M SLS in water at 37° C.±0.5° C.

62. The pharmaceutical composition according to claim 46, further comprising polyethylene glycol or a derivative thereof.

63. The pharmaceutical composition according to claim 62, wherein the fenofibrate is a non-micronized fenofibrate having a particle size greater than or equal to about 50 μm.

64. The pharmaceutical composition according to claim 62, wherein the polyethylene glycol or a derivative thereof comprises one or more of PEG 200, PEG 300, PEG 400, PEG 600, PEG 1000, PEG 4000, PEG 6000, PEG 8000, PEG 20000, polyglycolyzed glycerides, polyethylene glycol-polyoxyethylenes, polyethylene glycol polypropylenes, and polyethylene glycol-polyoxypropylenes.

65. The pharmaceutical composition according to claim 62, wherein the adsorbent and polyethylene glycol or a derivative thereof is alternately coated with non-micronized fenofibrate and a surfactant.

66. The pharmaceutical composition according to claim 65, wherein the surfactant comprises one or more of one or more of sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer, and cremophore RH 40.

67. The pharmaceutical composition according claim 46, which when administered to human subjects in the fed state at a dose of 145 mg exhibits (a) the mean area under the 96 hour AUC curve in the range from about 56.02 to about 268.23 (μg*hr/ml); (b) the mean area under the AUC curve extrapolated to infinite time in the range from about 59.07 to about 291.33 (μg*hr/ml); and (c) the maximum plasma concentration in the range from about 3.886 to about 20.703 μg/ml.
